(19) United States
(12) Patent Application Publication
(10) Pub. No.: US 2026/0207240 A1
(43) Pub. Date: Jul. 23, 2026

(54) ABNORMAL PULSE DELIVERY PROTECTION FOR PULSED FIELD ABLATION SYSTEMS

(71) Applicant: Medtronic, Inc., Minneapolis, MN (US)

(72) Inventors: Steven J. Fraasch, Maple Grove, MN (US); Vinicius Binotti, Elk River, MN (US); Jon E. Zimmer, Shoreview, MN (US)

(21) Appl. No.: 19/138,624

(22) PCT Filed: Nov. 28, 2023
(86) PCT No.: PCT/IB2023/061966

§ 371 Date: Jun. 12, 2025

Related U.S. Application Data
(60) Provisional application No. 63/387,473, filed on Dec. 14, 2022.

(51) Int. Cl. A61B 18/00

(52) U.S. Cl. CPC A61B 18/00; A61B 2018/00672; A61B 2018/00678; A61B 2018/00702; A61B 2018/0072; A61B 2018/00767; A61B 2018/00827; A61B 2018/00869; A61B 2018/00886

(57) ABSTRACT

One aspect provides a pulsed field ablation system including a bridge circuit configured to deliver bipolar and biphasic voltage pulses to a catheter. An asynchronous current monitor circuit electrically coupled the bridge circuit. An electronic processor is coupled to the bridge circuit and the asynchronous current monitor circuit. The electronic processor is configured to determine whether an abnormal pulse delivery is present in the bridge circuit and control the bridge circuit to deliver therapeutic current to the catheter when the abnormal pulse delivery is not present. The electronic processor is also configured to inhibit the bridge circuit from delivering therapeutic current to the catheter when the abnormal pulse delivery is present.

This application claims the benefit of U.S. Provisional Patent Application Ser. No. 63/387,473, filed Dec. 14, 2022, the entire content of which is incorporated herein by reference.

### BACKGROUND

Pulsed field ablation delivers a sequence of fast, bipolar, and biphasic high voltage pulses to perform irreversible electroporation of tissue. Pulsed field ablation is used to treat, among other things, cardiac arrhythmias and atrial fibrillation. Pulsed field ablation may also be used as an oncology treatment for cancer.

### SUMMARY

Electrophysiology procedures are used to treat a number of different conditions. A pulsed field ablation system may be used to deliver a sequence of fast, bipolar, and biphasic high voltage direct-current (DC) pulses to a patient to achieve irreversible electroporation. A catheter is used to deliver the high-voltage pulses to the patient. Extra ventricular contractions should be avoided during the procedure to avoid pain due to nerve stimulation and the need for patient sedation because of muscle activation. Delivering the biphasic pulses with a short duration between pulses helps reduce extra ventricular contractions. However, a pulsed field ablation system may sometimes fail and deliver monophasic pulses or long interval biphasic pulses which can cause extra ventricular contractions.

Accordingly, there is a need for abnormal pulse delivery protection in pulse field ablation systems.

The techniques disclosed herein generally relate to abnormal pulse delivery detection circuit and method for pulsed field ablation system. The abnormal pulse delivery detection circuit and method help to reduce undesirable or non-therapeutic current being delivered to a patient causing extra ventricular contractions.

One aspect provides a pulsed field ablation system including a bridge circuit configured to deliver bipolar and biphasic voltage pulses to a catheter, an asynchronous current monitor circuit electrically coupled the bridge circuit, and an electronic processor coupled to the bridge circuit and the asynchronous current monitor circuit. The electronic processor is configured to determine whether an abnormal pulse delivery is present in the bridge circuit and control the bridge circuit to deliver therapeutic current to the catheter when the abnormal pulse delivery is not present. The electronic processor is also configured to inhibit the bridge circuit from delivering therapeutic current to the catheter when the abnormal pulse delivery is present.

Another aspect provides a method for abnormal pulse delivery protection in a pulsed field ablation system including a bridge circuit configured to deliver bipolar and biphasic voltage pulses to a catheter and an asynchronous current monitor circuit coupled to the bridge circuit. The method includes determining, using the asynchronous current monitor circuit, whether an abnormal pulse delivery is present in the bridge circuit and controlling, using an electronic processor, the bridge circuit to deliver therapeutic current to the catheter when the abnormal pulse delivery is not present. The method also includes inhibiting, using the electronic processor, the bridge circuit from delivering therapeutic current to the catheter when the abnormal pulse delivery is present.

Various embodiments, examples, aspects, and features are set forth in the description below and the accompanying drawings. Other embodiments, examples, aspects, features, objects, and advantages of the techniques described in this disclosure will be apparent from the description and drawings, and from the claims.

### BRIEF DESCRIPTION OF DRAWINGS

The accompanying figures, where like reference numerals refer to identical or functionally similar elements throughout the separate views, which together with the detailed description below are incorporated in and form part of the specification and serve to further illustrate various embodiments, examples, aspects, and features that include the claimed subject matter, and to explain various principles and advantages of aspects of those embodiments, examples, aspects, and features.

FIG. 1 is a simplified block diagram that illustrates a pulsed field ablation system in accordance with some examples.

FIG. 2 illustrates voltage pulses delivered by the pulsed field ablation system of FIG. 1 in accordance with some examples.

FIG. 3 is a simplified schematic that illustrates a bridge circuit of the pulsed field ablation system of FIG. 1 in accordance with some examples.

FIG. 4 is a simplified block diagram that illustrates an asynchronous current monitor circuit of the pulsed field ablation system of FIG. 1 in accordance with some examples.

FIGS. 5A and 5B are flowcharts for a method for detecting abnormal pulse delivery in the pulsed field ablation system of FIG. 1 in accordance with some examples.

FIG. 6 is a flowchart for a method for abnormal pulse delivery protection in the pulsed field ablation system of FIG. 1 in accordance with some examples.

FIG. 7 is a flowchart for a method for abnormal pulse delivery protection in the pulsed field ablation system of FIG. 1 in accordance with some examples.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of examples.

The apparatus and method components have been represented where appropriate by conventional symbols in the drawings, showing only those specific details that are pertinent to understanding the embodiments, examples, aspects, and features so as not to obscure the disclosure with details that will be readily apparent to those of ordinary skill in the art having the benefit of the description herein.

### DETAILED DESCRIPTION

Before any embodiments, examples, aspects, and features are explained in detail, it is to be understood that those embodiments, examples, aspects, and features are not limited in their application to the details of construction and the arrangement of components set forth in the following description or illustrated in the following drawings. Other embodiments, examples, aspects, and features are possible and are capable of being practiced or carried out in various ways.

Also, it is to be understood that the phraseology and terminology used herein is for the purpose of description and should not be regarded as limiting. The terms “mounted,” “connected,” and “coupled” are used broadly and encompass both direct and indirect mounting, connecting, and coupling. The terms “connected” and “coupled” are not restricted to physical or mechanical connections or couplings, and can include electrical connections or couplings, whether direct or indirect. Electronic communications and notifications described herein may be performed using any known or future-developed means including wired connections, wireless connections, etc.

For ease of description, some or all of the example systems presented herein are illustrated with a single exemplar of each of its component parts. Some examples may not describe or illustrate all components of the systems. Other embodiments, examples, aspects, and features may include more or fewer of each of the illustrated components, may combine some components, or may include additional or alternative components.

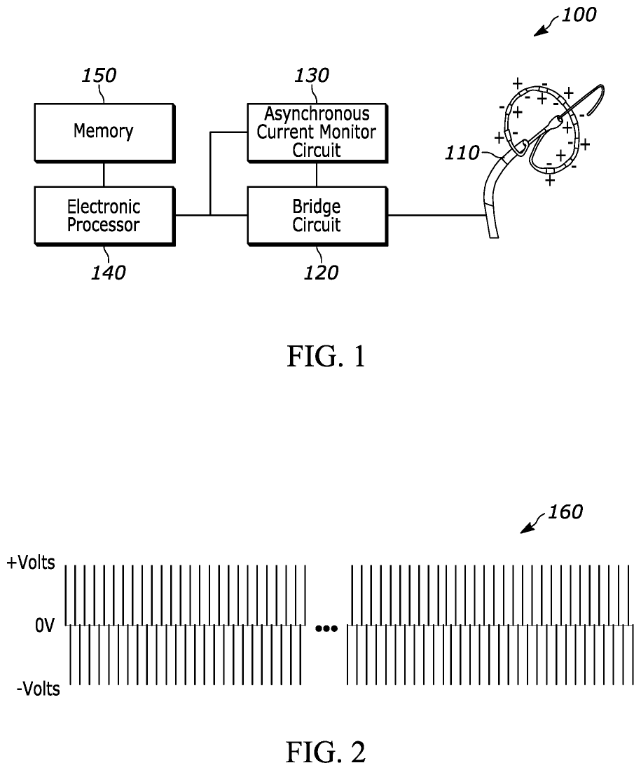

FIG. 1 illustrates a simplified block diagram of an example of a pulsed field ablation system 100. The pulsed field ablation system 100 is used to deliver a sequence of fast, bipolar, and biphasic voltage pulses (for example, as shown in FIG. 2) to a catheter 110 to perform irreversible electroporation of tissue. The pulsed field ablation system 100 includes a bridge circuit 120, an asynchronous current monitor circuit 130, an electronic processor 140, and a memory 150.

The catheter 110 is a multi-electrode catheter including a plurality of electrodes arranged successively around an enclosed or semi-enclosed area. The catheter 110 delivers the voltage pulses to tissue within the enclosed or semi-enclosed area. In some examples, the catheter 110 may be a disposable catheter 110 that is disposed after each use, while a new disposable catheter 110 is connected to the pulsed field ablation system 100 for every distinct procedure. The bridge circuit 120 is electrically coupled to the catheter 110. The bridge circuit 120 generates and delivers the voltage pulses to the catheter 110. The asynchronous current monitor circuit 130 is electrically coupled to the bridge circuit 120. The asynchronous current monitor circuit 130 detects abnormal pulses generated by the bridge circuit 120.

The electronic processor 140 is electrically coupled to the bridge circuit 120 and the asynchronous current monitor circuit 130 and is configured to control and monitor the bridge circuit 120 and the asynchronous current monitor circuit 130. In some examples, the electronic processor 140 is implemented as a microprocessor with separate memory, such as the memory 150. In other examples, the electronic processor 140 may be implemented as a microcontroller (with memory 150 on the same chip). In other examples, the electronic processor 140 may be implemented using multiple processors (in some cases located remote from one another). In addition, the electronic processor 140 may be implemented partially or entirely as, for example, a field-programmable gate array (FPGA), an applications specific integrated circuit (ASIC), an x86 processor, and the like and the memory 150 may not be needed or be modified accordingly. In the example, illustrated, the memory 150 includes non-transitory, computer readable memory that stores instructions that are received and executed by the electronic processor 140 to carry out the functionality of the pulsed field ablation system 100 described herein. The memory 150 may include, for example, a program storage area and a data storage area. The program storage area and the data storage area may include combinations of different types of memory, such as read-only memory and random-access memory. In some examples, the pulsed field ablation system 100 includes one electronic processor 140 and/or a plurality of electronic processors 140 in a computer cluster arrangement, one or more of which may be executing none, all, or a portion of the applications of the pulsed field ablation system 100.

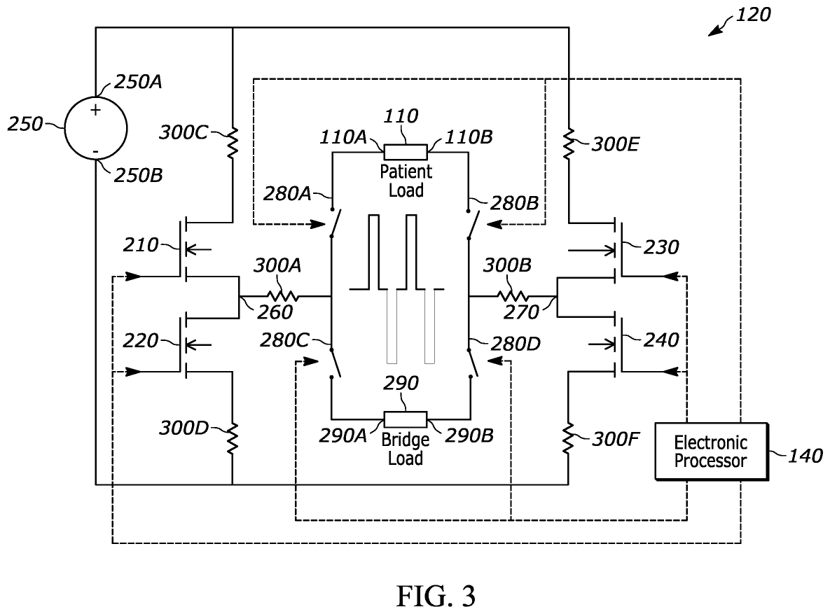

FIG. 3 illustrates a simplified schematic of the bridge circuit 120. In the example illustrated, the bridge circuit 120 is a full H-bridge circuit. In other examples, the bridge circuit 120 may be an inverter bridge circuit, or the like. The full H-bridge circuit is made up of a first transistor switch 210, a second transistor switch 220, a third transistor switch 230, and a fourth transistor switch 240. The transistor switches 210-240 include, for example, insulated-gate bipolar transistors (IGBTs), field effect transistors (FETs), and/or the like.

A power supply 250 provides high-voltage power to the H-bridge circuit. For pulsed field ablation systems 100, the high-voltage power may be in the range of between 300 Volts and 2000 Volts. The power supply 250 generates the high-voltage potential between a positive power supply node 250A and a negative power supply node 250B (for example, electric ground). The power supply 250 may include a high-voltage battery system or an alternating current (AC) power system that is converted to direct-current (DC) power.

The first transistor switch 210 is electrically coupled between the positive power supply node 250A and a first bridge output node 260. The second transistor switch 220 is electrically coupled between the first bridge output node 260 and the negative power supply node 250B. In one example, a source of the first transistor switch 210 is electrically coupled to a drain of the second transistor switch 220 at the first bridge output node 260. The third transistor switch 230 is electrically coupled between the positive power supply node 250A and a second bridge output node 270. The fourth transistor switch 240 is electrically coupled between the second bridge output node 270 and the negative power supply node 250B. In one example, a source of the third transistor switch 230 is electrically coupled to a drain of the fourth transistor switch 240 at the second bridge output node 270.

The bridge circuit 120 also includes a first patient cathode electrode connector 110A and a second patient cathode electrode connector 110B. The first patient cathode electrode connector 110A and the second patient cathode electrode connector 110B are configured to be connected to opposing electrodes (for example, positive and negative electrodes respectively) of the catheter 110 (for example, patient load) to deliver the voltage pulses from the bridge circuit 120. The first patient cathode electrode connector 110A and the second patient cathode electrode connector 110B are electrically coupled between the first bridge output node 260 and the second bridge output node 270. A first relay 280A is provided between the first bridge output node 260 and the first patient cathode electrode connector 110A and a second relay 280B is provided between the second patient cathode electrode connector 10B and the second bridge output node 270. The first relay 280A and the second relay 280B are controlled by the electronic processor 140 to selectively open and close the electrical path between the first bridge output node 260, the catheter 110, and the second bridge output node 270.

The bridge circuit 120 also includes a first patient-isolated internal load connector 290A and a second patient-isolated internal load connector 290B. The first patient-isolated internal load connector 290A and the second patient-isolated internal load connector 290B connect a patient-isolated internal load 290 between the first bridge output node 260 and the second bridge output node 270. The patient-isolated internal load 290 is used for detecting a leakage fault in the bridge circuit 120. A third relay 280C is provided between the first bridge output node 260 and the patient-isolated internal load 290 and a fourth relay 280D is provided between the patient-isolated internal load 290 and the second bridge output node 270. The third relay 280C and the fourth relay 280D are controlled by the electronic processor 140 to selectively open and close the electrical path between the first bridge output node 260, the patient-isolated internal load 290, and the second bridge output node 270.

A first resistor 300A is electrically coupled between (i) the first bridge output node 260 and (ii) the first relay 280A and the third relay 280C. A second resistor 300B is electrically coupled between (i) the second bridge output node 270 and (ii) the second relay 280B and the fourth relay 280D. A third resistor 300C is electrically coupled between the positive power supply node 250A and the first transistor switch 210. A fourth resistor 300D is electrically coupled between the second transistor switch 220 and the negative power supply node 250B. A fifth resistor 300E is electrically coupled between the positive power supply node 250A and the third transistor switch 230. A sixth resistor 300F is electrically coupled between the fourth transistor switch 240 and the negative power supply node 250B. The resistors 300A-F may be used as current detecting elements of the asynchronous current monitor circuit 130 as described in greater detail below.

The electronic processor 140 is used to control the transistor switches 210-240 and the relays 280 to selectively open and close the electrical paths respectively. A gate driver may be included in the bridge circuit 120 to provide driving signals to the transistor switches 210-240. The gate driver provides driving signals to the transistor switches 210-240 based on the control signals received from the electronic processor 140. When the transistor switches 210-240 are closed, the transistor switches 210-240 allow current to flow through the transistor switches 210-240 to components connected downstream of the transistor switches 210-240. When the transistor switches 210-240 are opened, the transistor switches 210-240 inhibit current flow through the transistor switches 210-240 to components connected downstream of the transistor switches 210-240. Similarly, when the relays 280 are closed, the relays 280 allow current to flow through the relays 280 to components connected downstream of the relays 280. When the relays 280 are opened, the relays 280 inhibit current flow through the relays 280 to components connected downstream of the relays 280.

The first relay 280A and the second relay 280B are closed to form an electrical path between the first bridge output node 260, the catheter 110, and the second bridge output node 270 for delivering therapeutic current to the catheter 110. The third relay 280C and the fourth relay 280D are opened when delivering the therapeutic current to the catheter 110. The electronic processor 140 controls the transistor switches 210-240 to provide sequential bipolar, biphasic high-voltage pulses to the catheter 110. The transistor switches 210-240 may be configured such that the transistor switches 210-240 are normally open. That is, the default state of the transistor switches 210-240 is an open state. The electronic processor 140 closes the first transistor switch 210 and the fourth transistor switch 240 and keeps the second transistor switch 220 and the third transistor switch 230 open to provide therapeutic current in a first direction (for example, a positive direction) to the catheter 110. The electronic processor 140 closes the second transistor switch 220 and the third transistor switch 230 and keeps the first transistor switch 210 and the fourth transistor switch 240 open to provide therapeutic current in a second direction (for example, a negative direction) to the catheter 110. The switching between the first direction and the second direction is performed at a high frequency. For example, the therapeutic current is provided in each direction for 4 microseconds with a 5 microsecond gap between each direction. During the 5 microsecond gap, all transistor switches 210-240 are turned off.

The bridge circuit 120 is designed to provide biphasic voltage pulses with very short duration (for example, 4 microseconds for pulsed field ablation). However, components of the bridge circuit 120 may sometimes fail resulting in abnormal voltage pulses being delivered to the catheter 110. Abnormal pulse delivery may include monophasic pulse delivery or long duration biphasic pulse delivery. These abnormal voltage pulses may cause undesirable extra ventricular contractions. The asynchronous current monitor circuit 130 may be used to detect abnormal pulses generated or delivered by the bridge circuit 120.

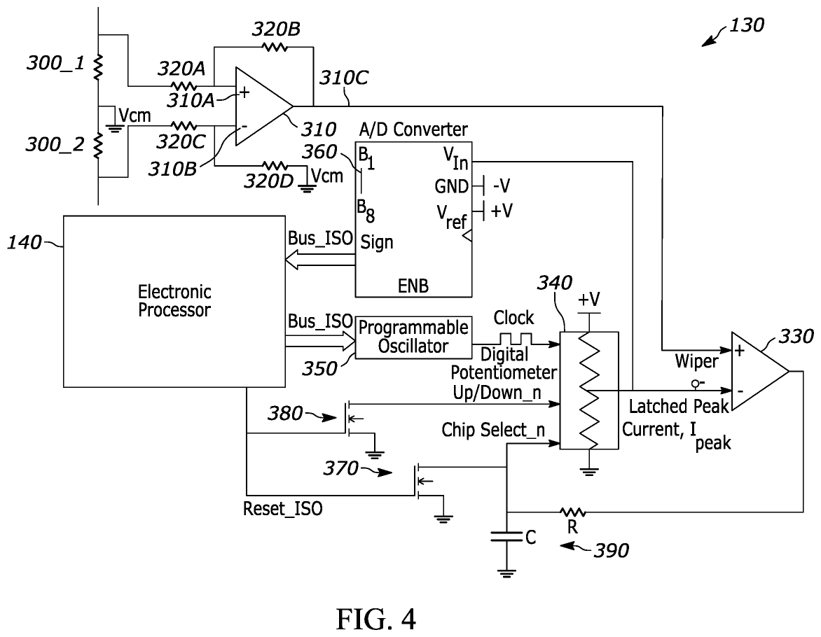

FIG. 4 illustrates a simplified schematic of the asynchronous current monitor circuit 130. The asynchronous current monitor circuit 130 may be connected across any one or more of the resistors 300 (for example, current detection element). In the example illustrated, the asynchronous current monitor circuit 130 includes series connected measurement resistors 300_1 and 300_2. The measurement resistors 300_1 and 300_2 represent any one of the resistors 300. A differential amplifier 310 is connected across the measurement resistors 300_1 and 300_2 such that a first end of the measurement resistors 300_1 and 300_2 is connected to the non-inverting input 310A of the differential amplifier 310 and a second end of the measurement resistors 300_1 and 300_2 is connected to the inverting input 310B of the differential amplifier 310. Resistors 320A-D are connected between the measurement resistors 300_1 and 300_2, the inputs 310A-B of the differential amplifier 310, and an output 310C of the differential amplifier 310 to provide a voltage gain. The resistors 320A-D may be selected to sufficiently amplify a current flowing through the current detection elements of the pulsed field ablation system 100.

The output 310C of the differential amplifier 310 is electrically coupled to non-inverting input of a threshold comparator 330. The asynchronous current monitor circuit 130 also includes a digital potentiometer 340 that provides a variable voltage output to the inverting input of the threshold comparator 330. A programmable oscillator 350 provides a clock signal to the digital potentiometer 340. A threshold output of the threshold comparator 330 is provided to an enable input of the digital potentiometer 340. The digital potentiometer 340 is configured for a voltage range between a minimum voltage and a maximum voltage. When the digital potentiometer 340 is enabled, for example, using the enable input, the voltage output of the digital potentiometer 340 varies between the minimum voltage and the maximum voltage based on an input clock signal and a control signal received from the electronic processor 140. In the example of FIG. 4, the enable input includes a chip select input, Chip_Select_n, where the n indicates inverted logic.

The voltage output of the digital potentiometer 340 is also provided to an analog to digital converter 360. The analog to digital converter 360 converts the analog voltage value detected at the voltage output to a digital value corresponding to the voltage value and provides the digital value to the electronic processor 140. The electronic processor 140 controls the programmable oscillator 350 to provide a clock signal to the digital potentiometer 340. A resetting circuit 370 is coupled between the electronic processor 140 and the enabling input of the digital potentiometer 340. An up/down control circuit 380 is coupled between the electronic processor 140 and a control input of the digital potentiometer 340. In the example of FIG. 4, the up/down control circuit provides an UP/DOWN_n signal to the digital potentiometer 340, where the n indicates inverted logic. For example, when the control signal is low, the voltage output of the digital potentiometer 340 increases from its current value to a higher value between the minimum voltage and the maximum voltage. When the control signal is high, the voltage output of the digital potentiometer 340 decreases from its current value to a lower value between the minimum voltage and the maximum voltage.

The threshold comparator 330 and the digital potentiometer 340 are configured to detect a peak current flowing through the resistor 300. When a current flows across the measurement resistors 300_1 and 300_2, the voltage drop across the measurement resistors 300_1 and 300_2 is amplified by the differential amplifier 310 and a detection parameter proportional to the voltage drop is provided to the non-inverting input of the threshold comparator 330. The threshold output of the threshold comparator 330 switches states (for example, from high to low or low to high) when the detection parameter at the non-inverting input exceeds the output of the digital potentiometer 340 at the inverting input. The threshold comparator 330 and the digital potentiometer 340 are also configured such that the digital potentiometer 340 is enabled by the threshold output when the voltage output of the digital potentiometer 340 is less than the detection parameter and is disabled by the threshold output when the voltage output is greater than the detection parameter. The digital potentiometer 340 is disabled by the threshold comparator 330 when the voltage output of the digital potentiometer 340 corresponds to (that is, equal to or just greater than) the detection parameter. The voltage output is latched at the voltage value, which is converted and provided to the electronic processor 140 by the analog to digital converter 360. The electronic processor 140 resets the digital potentiometer 340 using the resetting circuit 370 once the peak value is determined by the electronic processor 140.

In some examples, oscillations at the enabling input of the digital potentiometer 340 could trigger a write operation to an internal memory, which renders the digital potentiometer 340 unusable for a significant amount of time. To mitigate this unintended behavior, a timer circuit 390 is electrically coupled between the threshold output of the comparator 330 and the enabling input of the digital potentiometer 340. A time constant of the timer circuit 390 is configured to avoid an unintentional triggering of a write operation by the threshold output of the threshold comparator 330. In one example, the time constant of the timer circuit 390 is 0.82 microseconds.

FIGS. 1 and 4 illustrate a single asynchronous current monitor 130 for simplicity of explanation. However, the pulsed field ablation system 100 may include multiple asynchronous current monitors 130 connected across multiple resistors 300 to measure current flow in both directions. In one example, a first asynchronous current monitor 130 is connected across the first resistor 300A to measure current in the first direction and a second asynchronous current monitor 130 is also connected across the first resistor 330A to measure current in the second direction. In another example, a third asynchronous current monitor 130 is connected across the second resistor 300B to measure current in the first direction and a fourth asynchronous current monitor 130 is also connected across the second resistor 330B to measure current in the second direction. In some examples, the single asynchronous current monitor 130 may include multiple components, for example, a plurality of differential amplifiers 310, threshold comparators 330, and digital potentiometers 340 connected across the first resistor 300A and the second resistor 300B. For example, first and second differential amplifiers 310, first and second threshold comparators 330, and first and second digital potentiometers 340 may be connected across the first resistor 300A to detect current flowing in the first direction and the second direction respectively. Similarly, third and fourth differential amplifiers 310, third and fourth threshold comparators 330, and third and fourth digital potentiometers 340 may be connected across the second resistor 300B to detect current flowing in the first direction and the second direction respectively.

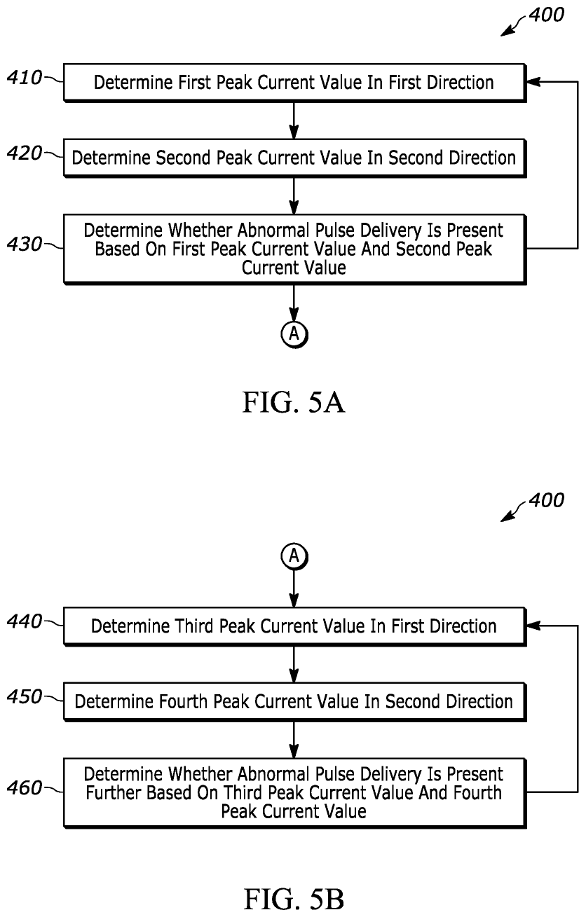

FIG. 5A is a flowchart of an example method 400 for detecting abnormal pulse delivery in the pulsed field ablation system 100. In the example illustrated, the method 400 includes determining a first peak current value in the first direction (at block 410) and determining a second peak current value in the second direction (at block 420). In one example, the electronic processor 140 uses the first asynchronous current monitor 130 to detect the first peak current value in the first direction and uses the second asynchronous current monitor 130 to detect the second peak current value in the second direction. In another example, the electronic processor 140 uses the first digital potentiometer 340 to detect the first peak current value in the first direction and uses the second digital potentiometer 340 to detect the second peak current value in the second direction. The first peak current value and the second peak current value are detected for a first current detection element (for example, the first resistor 300A)

The method 400 includes determining whether an abnormal pulse delivery is present based on the first peak current value and the second peak current value (at block 430). In one example, the electronic processor 140 calculates a monophasic asymmetry index (MAI) using the following formula:

$$MAI = \frac{\vert {I}_{1} - {I}_{2} \vert}{{I}_{1} + {I}_{2}}$$

where I₁ is the first peak current value and I₂ is the second peak current value. An MAI of 1 indicates total asymmetry, that is, ideal monophasic pulses. An MAI of 0 indicates total symmetry, that is, ideal biphasic pulses. Any value above 0 or above a suitable fractional or decimal threshold between 0 and 1 may indicate an abnormal pulse delivery.

In some examples, the pulsed field ablation system 100 may optionally use two current detection elements (for example, the first resistor 300A and the second resistor 300B) to detect current flow on either side of the catheter 110 to determine whether an abnormal pulse delivery is present. FIG. 5B illustrates an extension of method 400 to determine whether the abnormal pulse delivery is present based on two current detection elements. In the example illustrated, the method 400 includes determining a third peak current value in the first direction (at block 440) and determining a fourth peak current value in the second direction (at block 450). In one example, the electronic processor 140 uses the third asynchronous current monitor 130 to detect the third peak current value in the first direction and uses the fourth asynchronous current monitor 130 to detect the fourth peak current value in the second direction. In another example, the electronic processor 140 uses the third digital potentiometer 340 to detect the third peak current value in the first direction and uses the fourth digital potentiometer 340 to detect the fourth peak current value in the second direction. The third peak current value and the fourth peak current value are detected for a second current detection element (for example, the second resistor 300B)

The method 400 includes determining whether an abnormal pulse delivery is present further based on the third peak current value and the fourth peak current value (at block 460). In one example, the electronic processor 140 calculates the monophasic asymmetry index (MAI) using the following formula:

$$MAI = \frac{\frac{\vert {I}_{1} - {I}_{2} \vert}{{I}_{1} + {I}_{2}} + \frac{\vert {I}_{3} - {I}_{4} \vert}{{I}_{3} + {I}_{4}}}{2}$$

where I₃ is the third peak current value and I₄ is the fourth peak current value. An MAI of 1 indicates total asymmetry, that is, ideal monophasic pulses. An MAI of 0 indicates total symmetry, that is, ideal biphasic pulses. Any value above 0 or above a suitable fractional or decimal threshold between 0 and 1 may indicate an abnormal pulse delivery.

The period between each successive calculation of the MAI by the electronic processor 140 can vary from one biphasic/monophasic pulse to the entire pulse train. The electronic processor 140 may vary the period for calculating the MAI (for example, to determine the abnormal pulse delivery) based on user input or based on the pulse field ablation system 100 requirements. However, the asynchronous current monitor may need to be reset after the computation to detect subsequent monophasic pulses that may occur before the end of the pulse train. Depending on the duration and number of pulses over which the MAI would be calculated, a threshold for the MAI can be defined based on empirical data. Having defined the threshold, the method 400 uses the calculated MAI to determine whether a therapy delivery has monophasic pulses. For the evaluation of the method 400, the single-sided MAI (for example, the MAI determined at block 430) may be computed every 900 microseconds along with a threshold of 0.76, and a pulse of 200 microseconds to reset the digital potentiometer.

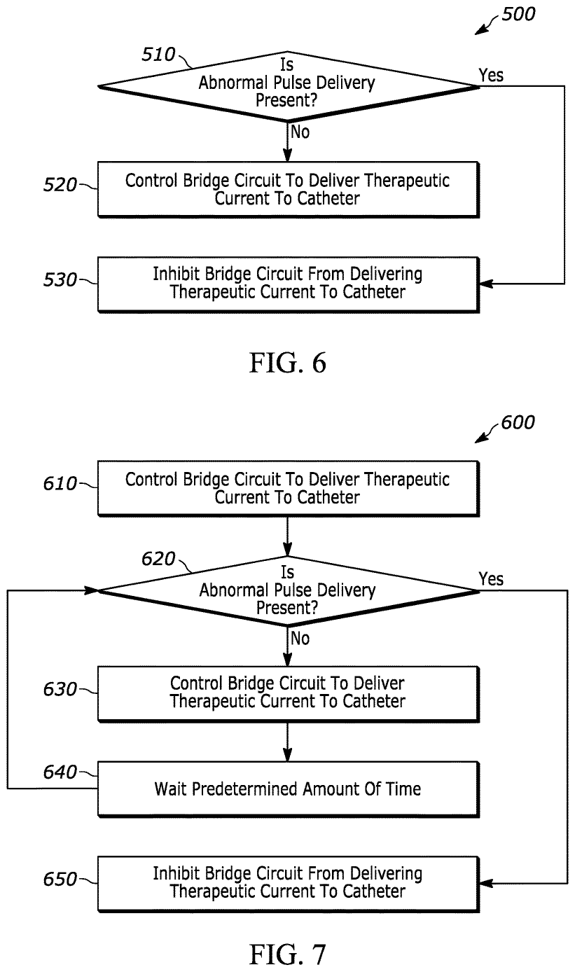

FIG. 6 is a flowchart of an example method 500 for abnormal pulse delivery protection in the pulsed field ablation system 100. In the example illustrated, the method 500 includes determining, using the asynchronous current monitor circuit 130, whether an abnormal pulse delivery is present in the pulsed field ablation system 100 (at block 510). The electronic processor 140 uses the method 400 of FIG. 5A and/or FIG. 5B to determine whether an abnormal pulse deliver is present in the pulsed field ablation system 100.

The method 500 includes controlling, using the electronic processor 140, the bridge circuit 120 to deliver therapeutic current to the catheter 110 when an abnormal pulse delivery is not present (at block 520). In response to determining that an abnormal pulse delivery is not present based on the MAI, the electronic processor 140 operates the bridge circuit 120 normally to produce the therapeutic current and provide the therapeutic current to the catheter 110. The method 500 includes inhibiting, using the electronic processor 140, the bridge circuit 120 from delivering therapeutic current to the catheter 110 when an abnormal pulse delivery is present (at block 530). In response to determining that an abnormal pulse delivery is present based on the MAI, the electronic processor 140 may turn off the bridge circuit 120 and inhibit the bridge circuit 120 from providing therapeutic current to the catheter 110.

FIG. 7 is a flowchart of an example method 600 for abnormal pulse delivery protection in the pulsed field ablation system 100. The method 600 is similar to method 500 and may be performed concurrently with method 500 to provide abnormal pulse delivery protection. In the example illustrated, the method 600 including controlling, using the electronic processor 140, the bridge circuit 120 to delivery therapeutic current to the catheter 110 (at block 610). The electronic processor 140 operates the bridge circuit 120 normally to produce the therapeutic current and provide the therapeutic current to the catheter 110. The method 600 also includes determining, using the asynchronous current monitor circuit 130, whether an abnormal pulse delivery is present in the pulsed field ablation system 100 (at block 620). The electronic processor 140 uses the method 400 of FIG. 5A and/or FIG. 5B to determine whether an abnormal pulse deliver is present in the pulsed field ablation system 100. In some examples, the method 600 may implement an initial delay before commencing abnormal pulse delivery detection.

The method 600 includes resetting, using the electronic processor 140, the asynchronous current monitor circuit 130 when an abnormal pulse delivery is not present (at block 630). In response to determining that an abnormal pulse delivery is not present based on the MAI, the electronic processor 140 resets the asynchronous current monitor circuit 130 using the resetting circuit 370. After the asynchronous current monitor circuit 130 is reset, the method 600 includes waiting a predetermined amount of time (at block 640) before proceeding to perform the next instance of determining whether an abnormal pulse deliver is present at block 620. The predetermined time may be determined using the settling time. In one example, the predetermined time is 900 microseconds. The method 600 continues to deliver the therapeutic current as long as an abnormal pulse is not detected. The method 600, however, resets the asynchronous current monitor after every instance of current monitoring to reset the digital potentiometer 340. The method 600 includes inhibiting, using the electronic processor 140, the bridge circuit 120 from delivering therapeutic current to the catheter 110 when an abnormal pulse delivery is present (at block 650). In response to determining that an abnormal pulse delivery is present based on the MAI, the electronic processor 140 may turn off the bridge circuit 120 and inhibit the bridge circuit 120 from providing therapeutic current to the catheter 110.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

The following examples are a non-limiting list of clauses in accordance with one or more techniques of this disclosure.
Example 1. A pulsed field ablation system, comprising: a bridge circuit configured to deliver bipolar and biphasic voltage pulses to a catheter; an asynchronous current monitor circuit electrically coupled the bridge circuit; and an electronic processor coupled to the bridge circuit and the asynchronous current monitor circuit configured to determine whether an abnormal pulse delivery is present in the bridge circuit; control the bridge circuit to deliver therapeutic current to the catheter when the abnormal pulse delivery is not present; and inhibit the bridge circuit from delivering therapeutic current to the catheter when the abnormal pulse delivery is present.
Example 2. The pulsed field ablation system of Example 1, wherein the abnormal pulse delivery includes one or more selected from a group consisting of monophasic pulse delivery and long-duration biphasic pulse delivery.
Example 3. The pulsed field ablation system of any of the preceding claims, wherein the electronic processor is configured to determine a first peak current value in a first direction; determine a second peak current value in a second direction; and determine whether the abnormal pulse delivery is present based on the first peak current value and the second peak current value.
Example 4. The pulsed field ablation system of Example 3, wherein the electronic processor is further configured to determine a third peak current value in the first direction; determine a fourth peak current value in the second direction; determine whether the abnormal pulse delivery is present further based on the third peak current value and the fourth peak current value.
Example 5. The pulsed field ablation system of any of the preceding claims, wherein the asynchronous current monitor circuit includes a digital potentiometer configured to provide an output proportional to a current flowing through a current detection element of the bridge circuit, wherein the electronic processor is electrically coupled to the digital potentiometer and configured to receive the output from the digital potentiometer; and determine a peak current value flowing through the current detection element based on the output.
Example 6. The pulsed field ablation system of Example 5, further comprising: an analog to digital converter electrically coupled between the output of the digital potentiometer and the electronic processor, the analog to digital converter is configured to convert an analog voltage value received from the digital potentiometer to a digital value provided to the electronic processor.
Example 7. The pulsed field ablation system of any of Examples 5-6, wherein the asynchronous current monitor circuit further includes a threshold comparator configured to receive, at a non-inverting input, a detection parameter corresponding to the current flowing through the current detection element; receive, at an inverting input, the output of the digital potentiometer; and provide a threshold output to the digital potentiometer, wherein the digital potentiometer is configured to be enabled by the threshold output when the output is less than the detection parameter and is configured to be disabled by the threshold output when the output is greater than the detection parameter.
Example 8. The pulsed field ablation system of Example 7, wherein the asynchronous current monitor circuit further includes a differential amplifier electrically coupled between the current detection element and the threshold comparator, wherein the differential amplifier is configured to detect a voltage drop across the current detection element and provide the detection parameter to the threshold comparator, wherein the detection parameter is proportional to the voltage drop across the current detection element.
Example 9. The pulsed field ablation system of any of Examples 7-8, wherein the asynchronous current monitor circuit further includes a timer circuit electrically coupled between the threshold comparator and the digital potentiometer.
Example 10. The pulsed field ablation system of any of Examples 5-9, wherein the asynchronous current monitor circuit includes a second digital potentiometer configured to provide a second output proportional to a current flowing through a second current detection element of the bridge circuit wherein the electronic processor is electrically coupled to the second digital potentiometer and configured to receive the second output from the second digital potentiometer; and determine a second peak current value flowing through the second current detection element based on the second output.
Example 11. The pulsed field ablation system of any of Examples 5-10, wherein the bridge circuit includes a first transistor switch electrically coupled between a positive power supply node and a first bridge output node; a second transistor switch electrically coupled between the first bridge output node and a negative power supply node; a third transistor switch electrically coupled between the positive power supply node and a second bridge output node; and a fourth transistor switch electrically coupled between the second bridge output node and the negative power supply node, wherein the electronic processor is electrically coupled to and controls to selectively open and close the first transistor switch, the second transistor switch, the third transistor switch, and the fourth transistor switch.
Example 12. The pulsed field ablation system of Example 11, wherein the current detection element is electrically coupled between the first bridge output node and the second bridge output node.
Example 13. The pulsed field ablation system of Example 12, wherein the first direction is a direction of current flow from the first transistor switch to the fourth transistor switch and the second direction is a direction of current flow from the third transistor switch to the second transistor switch.
Example 14. The pulsed field ablation system of any of the preceding claims, wherein the electronic processor is further configured to vary a period for determining the abnormal pulse delivery.
Example 15. A method for abnormal pulse delivery protection in a pulsed field ablation system including a bridge circuit configured to deliver bipolar and biphasic voltage pulses to a catheter and an asynchronous current monitor circuit coupled to the bridge circuit, the method comprising: determining, using the asynchronous current monitor circuit, whether an abnormal pulse delivery is present in the bridge circuit; controlling, using an electronic processor, the bridge circuit to deliver therapeutic current to the catheter when the abnormal pulse delivery is not present; and inhibiting, using the electronic processor, the bridge circuit from delivering therapeutic current to the catheter when the abnormal pulse delivery is present.
Example 16. The method of Example 15, wherein the abnormal pulse delivery includes one or more selected from a group consisting of monophasic pulse delivery and long-duration biphasic pulse delivery.
Example 17. The method of any of Examples 15-16, further comprising: determining a first peak current value in a first direction; determining a second peak current value in a second direction; and determining whether the abnormal pulse delivery is present based on the first peak current value and the second peak current value.
Example 18. The method of Example 17, further comprising: determining a third peak current value in the first direction; determining a fourth peak current value in the second direction; determining whether the abnormal pulse delivery is present further based on the third peak current value and the fourth peak current value.
Example 19. The method of any of Examples 15-18, further comprising: providing, using a digital potentiometer of the asynchronous current monitor circuit, an output proportional to a current flowing through a current detection element of the bridge circuit; determining a peak current value flowing through the current detection element based on the output.
Example 20. The method of Example 19, further comprising converting, using an analog to digital converter, an analog voltage value received from the digital potentiometer to a digital value.
Example 21. The method of any of Examples 19-20, further comprising: receive, using a threshold comparator of the asynchronous current monitor circuit, a detection parameter corresponding to the current flowing through the current detection element; receiving, using the threshold comparator, the output of the digital potentiometer; enabling, using the threshold comparator, the digital potentiometer when the output is less than the detection parameter and disabling, using the threshold comparator, the digital potentiometer when the output is greater than the detection parameter.
Example 22. The method of Example 21, further comprising: detecting, using a differential amplifier, a voltage drop across the current detection element; and providing, using the differential amplifier, the detection parameter to the threshold comparator, wherein the detection parameter is proportional to the voltage drop across the current detection element.
Example 23. The method of any of Examples 18-22, further comprising: providing, using a second digital potentiometer, a second output proportional to the current flowing through a second current detection element of the bridge circuit; and determining a second peak current value flowing through the second current detection element based on the second output.
Example 24. The method of any of Examples 15-23, further comprising: varying a period for determining the abnormal pulse delivery.

What is claimed is:

1. A pulsed field ablation system, comprising:
a bridge circuit configured to deliver bipolar and biphasic voltage pulses to a catheter;
an asynchronous current monitor circuit electrically coupled to the bridge circuit; and
an electronic processor coupled to the bridge circuit and the asynchronous current monitor circuit configured to:
determine that an abnormal pulse delivery is not present in the bridge circuit;
control the bridge circuit to deliver therapeutic current to the catheter in response to determining that the abnormal pulse delivery is not present;
determine that the abnormal pulse delivery is present in the bridge circuit and
inhibit the bridge circuit from delivering therapeutic current to the catheter in respond to determining that the abnormal pulse delivery is present.

2. The pulsed field ablation system of claim 1, wherein the abnormal pulse delivery includes one or more selected from a group consisting of monophasic pulse delivery and long-duration biphasic pulse delivery.

3. The pulsed field ablation system of claim 1, wherein the electronic processor is configured to:
determine a first peak current value in a first direction;
determine a second peak current value in a second direction; and
determine whether the abnormal pulse delivery is present based on the first peak current value and the second peak current value.

4. The pulsed field ablation system of claim 3, wherein the electronic processor is further configured to:
determine a third peak current value in the first direction;
determine a fourth peak current value in the second direction; and
determine whether the abnormal pulse delivery is present further based on the third peak current value and the fourth peak current value.

5. The pulsed field ablation system of claim 1, wherein the asynchronous current monitor circuit includes a digital potentiometer configured to provide an output proportional to a current flowing through a current detection element of the bridge circuit, wherein the electronic processor is electrically coupled to the digital potentiometer and configured to:
receive the output from the digital potentiometer; and
determine a peak current value flowing through the current detection element based on the output.

6. The pulsed field ablation system of claim 5, further comprising:
an analog to digital converter electrically coupled between the output of the digital potentiometer and the electronic processor, the analog to digital converter is configured to convert an analog voltage value received from the digital potentiometer to a digital value provided to the electronic processor.

7. The pulsed field ablation system of claim 5, wherein the asynchronous current monitor circuit further includes
a threshold comparator configured to
receive, at a non-inverting input, a detection parameter corresponding to the current flowing through the current detection element;
receive, at an inverting input, the output of the digital potentiometer; and
provide a threshold output to the digital potentiometer,
wherein the digital potentiometer is configured to be enabled by the threshold output when the output is less than the detection parameter and is configured to be disabled by the threshold output when the output is greater than the detection parameter.

8. The pulsed field ablation system of claim 7, wherein the asynchronous current monitor circuit further includes
a differential amplifier electrically coupled between the current detection element and the threshold comparator, wherein the differential amplifier is configured to detect a voltage drop across the current detection element and provide the detection parameter to the threshold comparator, wherein the detection parameter is proportional to the voltage drop across the current detection element.

9. The pulsed field ablation system of claim 7, wherein the asynchronous current monitor circuit further includes a timer circuit electrically coupled between the threshold comparator and the digital potentiometer.

10. The pulsed field ablation system of claim 5, wherein the asynchronous current monitor circuit includes a second digital potentiometer configured to provide a second output proportional to a current flowing through a second current detection element of the bridge circuit wherein the electronic processor is electrically coupled to the second digital potentiometer and configured to
receive the second output from the second digital potentiometer; and
determine a second peak current value flowing through the second current detection element based on the second output.

11. The pulsed field ablation system of claim 5, wherein the bridge circuit includes:
a first transistor switch electrically coupled between a positive power supply node and a first bridge output node;
a second transistor switch electrically coupled between the first bridge output node and a negative power supply node;
a third transistor switch electrically coupled between the positive power supply node and a second bridge output node; and
a fourth transistor switch electrically coupled between the second bridge output node and the negative power supply node,
wherein the electronic processor is electrically coupled to and controls to selectively open and close the first transistor switch, the second transistor switch, the third transistor switch, and the fourth transistor switch.

12. The pulsed field ablation system of claim 11, wherein the current detection element is electrically coupled between the first bridge output node and the second bridge output node.

13. The pulsed field ablation system of claim 12, wherein the first direction is a direction of current flow from the first transistor switch to the fourth transistor switch and the second direction is a direction of current flow from the third transistor switch to the second transistor switch.

14. The pulsed field ablation system of claim 1, wherein the electronic processor is further configured to:
vary a period for determining the abnormal pulse delivery.
